(19) United States
(12) Patent Application Publication
(10) Pub. No.: US 2025/0136527 A1
(43) Pub. Date: May 1, 2025

(54) SYSTEM AND METHOD FOR UTILIZING RENEWABLE ELECTRICITY BY METHANOL SYNTHESIS VIA PLASMA-CATALYSIS CARBON DIOXIDE HYDROGENATION

(71) Applicant: ZHEJIANG UNIVERSITY, Hangzhou (CN)

(72) Inventors: Jianhua YAN, Hangzhou (CN); Hao ZHANG, Hangzhou (CN); Angjian WU, Hangzhou (CN); Kaiyi WANG, Hangzhou (CN)

(21) Appl. No.: 18/895,290

(22) Filed: Sep. 24, 2024

Related U.S. Application Data
(63) Continuation of application No. PCT/CN2024/075384, filed on Feb. 2, 2024.

(30) Foreign Application Priority Data
Oct. 30, 2023 (CN) 202311418656.0

(51) Int. Cl. C07C 1/04; C01B 32/40; C07C 1/12

(52) U.S. Cl. CPC C07C 1/042; C01B 32/40; C07C 1/041; C07C 1/12; C01B 2203/061

(57) ABSTRACT

A system and method for utilizing renewable electricity by methanol synthesis via plasma-catalysis CO₂ hydrogenation. Hydrogen produced via water electrolysis and CO₂ captured from industrial processes undergo a reverse water-gas shift reaction, driven efficiently by an atmospheric-pressure plasma jet, yielding a CO/CO₂/H₂ mixed product. This mixture is subsequently pressurized in multi-stage pressurization after passing a buffer storage tank to further efficiently synthesize green methanol in the plasma jet reactor. The present disclosure employs a two-stage methanol synthesis process that powered by renewable electricity: plasma-based CO₂ pre-conversion followed by CO/CO₂ catalytic hydrogenation. This approach addresses the issues of catalyst deactivation and high reaction temperatures associated with traditional thermocatalytic reverse water-gas shift reactions, while overcoming the thermodynamic limitations of direct CO₂ hydrogenation. The plasma jet reactor exhibits high energy efficiency, with rapid start-up and shutdown capabilities, and can operate directly using fluctuating renewable energy.

### CROSS-REFERENCE TO RELATED APPLICATIONS

This application is a continuation of International Application No. PCT/CN2024/075384, filed on Feb. 2, 2024, which claims priority to Chinese Application No. 202311418656.0, filed on Oct. 30, 2023, the contents of both of which are incorporated herein by reference in their entireties.

### TECHNICAL FIELD

The present disclosure belongs to the field of greenhouse gas utilization and renewable energy utilization or consumption, and relates to system and method for utilizing renewable electricity by methanol synthesis via plasma-catalysis CO₂ hydrogenation.

### BACKGROUND

The catalytic hydrogenation of CO₂ for methanol synthesis is a prominent research focus internationally. CO₂ is the major greenhouse gas with a wide range of sources, while green hydrogen can be readily obtained through water electrolysis by renewable energy. Methanol, a high value-added product, is liquid with the potential to directly utilize existing transportation and power infrastructure. It boasts high energy density, clean and efficient combustion, making it an ideal green fuel or hydrogen carrier. Additionally, methanol is the fourth largest basic chemical feedstock, capable of being converted into hundreds of chemical products. With the rise of renewable energy and the significant reduction in investment and operating costs, CO₂ hydrogenation for methanol synthesis driven by renewable energy has attracted significant attention.

Research and development into methanol synthesis from CO₂ hydrogenation under thermocatalytic conditions began early. In 1993, Lurgi launched the world's first demonstration project for CO₂ hydrogenation to methanol. In China, Dalian Institute of Chemical Physics, Chinese Academy of Sciences, Shanghai Institute of Advanced Research and others have conducted extensive industrial research. For instance, a skid-mounted device developed by the Dalian Institute of Chemical Physics and PetroChina has achieved a CO₂ conversion rate of 20% and methanol selectivity of 70%.

However, thermocatalytic CO₂ hydrogenation for methanol synthesis faces several challenges: Thermodynamic and kinetic limitations results in a low single-pass conversion rate (generally <20%) and require operation under high pressure conditions (generally >5 Mpa). Additionally, the catalytic process is complex with low flexibility, long response times (typically on the order of hours), and difficulty adapting to the significant variability of renewable electricity, limiting its scalability and broader application.

Therefore, there is a need for a method and system that can overcome these challenges, enabling low-energy, flexible, and controlled conversion of CO₂ to methanol, while accommodating the fluctuations of renewable electricity. Such advancement would greatly enhance the application prospects of renewable energy-driven CO₂ hydrogenation, enabling efficient chemical energy storage, greater utilization of renewable energy and the collaborative conversion of greenhouse gas CO₂. This would offer a transformative pathway to achieving carbon peak and carbon neutrality goals.

### SUMMARY

The present disclosure aims to provide a system and method for utilizing renewable electricity by methanol synthesis via plasma-catalysis CO₂ hydrogenation, addressing the shortcomings of prior art.

In a first aspect, a system for utilizing renewable electricity by methanol synthesis via plasma-catalysis CO₂ hydrogenation is provided, including a plasma jet reaction tower, a methanol synthesis reactor, a feed system, a storage and pressurization system, a power source and a purification treatment system.

The feed system is configured to provide H₂ and CO₂ for a plasma jet reactor.

The plasma jet reaction tower is configured to pre-activate and convert CO₂ by using plasma jet, causing CO₂ and H₂ to undergo reverse water-gas shift reaction, producing a CO/CO₂/H₂ mixed intermediate product at atmospheric pressure and low temperature.

The storage and pressurization system, comprising a mixed gas intermediate storage tank and a multi-stage pressurization system, is configured to store and pressurize the CO/CO₂/H₂ mixed gas produced by the plasma jet reactor.

The methanol synthesis reactor is configured to catalytically synthesize methanol from the CO/CO₂/H₂ mixed gas conveyed by the storage and pressurization system.

The power source is configured to supply renewable electricity to the plasma jet reactor, the methanol synthesis reactor, and the remaining system.

The purification treatment system is configured to separate methanol from the remaining reaction gas and purify the methanol product.

Further, the feed system includes a water electrolysis hydrogen production reactor, a H₂ storage tank, a CO₂ storage tank, a water pump, a first flow controller and a heat exchanger. The water pump, the first flow controller and the heat exchanger are configured to convey H₂O to the water electrolysis hydrogen production reactor and control flow, and the water electrolysis hydrogen production reactor is connected to the H₂ storage tank.

Further, each output of the H₂ storage tank, the CO₂ storage tank and the mixed gas intermediate storage tank is equipped with a check valve, a flow controller and a centrifugal pump.

Further, the top of the plasma jet reaction tower is provided with a gas collecting unit that directs gas to the mixed gas intermediate storage tank, while the bottom has a liquid collecting pipeline. Three vertically staggered plasma jet reaction groups are mounted on one side of the tower body, with each group consisting of six symmetrically arranged plasma jet reactors in a hexagonal array, fixed to a disk-shaped base. On the opposite side of the tower, three sight glasses allow observation of the reaction process, and temperature and pressure sensors are installed on the tower's surface.

Further, each plasma jet reactor includes an outer electrode, an inner electrode, a CO₂/H₂ inlet and a base.

The outer electrode, fixed to an insulating base, is a hollow sleeve electrode with a tapered outlet at an upper portion.

The inner electrode, conical in shape, is positioned at the lower-mid part of the hollow structure of the outer electrode, and integrally formed by a lower cylinder and an upper cone frustum. The inner electrode is fixed to the base and connected to a high voltage plasma power source via an electrode lead penetrating through the base; an outer wall of the inner electrode is parallel to an inner wall of the outer electrode; the H₂/CO₂ inlet is formed in a wall surface of the outer electrode, a H₂/CO₂ stream inlet is provided at a bottom of the plasma jet reactor and tangentially introduced from the bottom to form a swirling rising flow in a gap between the inner electrode and the outer electrode, to drive an arc between the inner electrode and outer electrodes to rise rotationally, and plasma is ejected in a jet form under the action of the tapered outlet. The outer and inner electrodes are connected to a frequency-adjustable high voltage alternating current (AC) power source.

Further, the methanol synthesis reactor is provided with a CO/CO₂ hydrogenation methanol synthesis reaction unit and a crude methanol outlet. CO/CO₂ and H₂ are selectively converted into crude methanol over copper-based composite nanocatalyst, which is then purified through the purification system. This enables efficient chemical energy storage of surplus/valley electricity and the collaborative conversion of greenhouse gas CO₂.

Further, the multi-stage pressurization system includes a booster pump, a buffer tank, a back pressure valve and a real-time pressure monitoring system.

Further, the purification treatment system includes: a reheater, a gas-liquid desuperheater, a flash tank, a methanol rectification tower and a methanol storage tank connected to one another in sequence. A liquid channel of the flash tank directly connects to the rectification tower, while the gas channel of the flash tank is connected to the methanol synthesis reactor and the methanol rectification tower. The purification treatment system is configured to separate the methanol from the remaining reaction gas and purify the methanol.

Further, the power source includes a water electrolysis hydrogen production power source, a plasma jet reactor power source and a multi-stage pressurization system power source, which are configured to supply power to the water electrolysis hydrogen production reactor, the plasma jet reactor and the multi-stage pressurization system respectively, all powered by off-grid renewable energy.

In a second aspect, a method for utilizing renewable electricity by methanol synthesis via plasma-catalysis CO₂ hydrogenation is provided, including:

Step 1, activating the power source for water electrolysis to produce hydrogen, and directly introducing the produced hydrogen into the H₂ storage tank for storage.

Step 2, opening the CO₂ storage and H₂ storage tank, mixing the gases in a specific ratio and introducing them into the plasma jet reactor, forming a swirling flow inside the reactor; activating the power source to form an arc between the inner and outer electrodes which then rises rotationally, ejecting plasma jet under the action of a tapered outlet. The reactor's power and gas flow are adjusted for optimal performance, while real-time temperature monitoring is conducted. CO₂ undergoes pre-activation and the following reaction occurs:

CO₂+H₂═CO+H₂O.

Step 3, the mixed gas produced by the reaction is over rising after passing an air distributor, introducing the mixed gas into a mixed gas intermediate storage tank for storage, and water flows out from a lower liquid collecting pipeline.

Step 4, the mixed gas is pressurized to 3-5 MPa using the multi-stage pressurization system and introduced into the methanol synthesis reactor, where it undergoes catalytic conversion into methanol.

Step 5, sequentially cooling, by a heat exchanger and a gas-liquid desuperheater, a gas-liquid mixture of the methanol produced after reaction in the methanol synthesis reactor and unreacted gas, performing gas-liquid separation on the cooled gas-liquid mixture using a flash tank, purifying, by a methanol rectification tower, separated liquid methanol, sending the purified liquid methanol to a methanol storage tank, passing separated gas through a separator, collecting and uniformly treating a part of the separated gas, and returning another part of the separated gas to the methanol synthesis reactor for further reaction.

Compared with the prior art, the present disclosure has the following advantages:

(1) Methanol is synthesized using a two-stage CO₂ hydrogenation process, overcoming the thermodynamic bottlenecks of direct CO₂ hydrogenation. Plasma efficiently pre-activates and partially converts CO₂ at atmospheric pressure, producing CO/CO₂/H₂ that can be more efficiently synthesized into methanol in both kinetic and thermodynamic terms, thereby remarkably improving the overall performance.

(2) CO₂ is activated by the plasma jet at atmospheric pressure through high-energy electrons and excited-state reactive species, facilitating the reverse water-gas shift reaction with H₂. No catalyst is needed during the reaction process, addressing issues of catalyst deactivation and high reaction temperatures in traditional thermocatalytic reverse water-gas shift reaction.

(3) The fractions of CO/CO₂/H₂ in the outlet mixed gas can be flexibly adjusted by regulating the power, the gas flow rate and other parameters of the plasma jet reactors, thereby optimizing the selectivity and the conversion rate of the catalytic methanol synthesis stage.

(4) A storage system decouples the plasma reactors from the methanol synthesis reactor, mitigating the impact of fluctuations in plasma reactions on methanol synthesis reaction.

(5) The entire system is powered by renewable energy. Plasma's rapid start/stop capability and fast reaction rates make it well-suited to the intermittency of renewable power sources, offering strong application potential in regions with abundant wind and solar energy, such as Inner Mongolia, China.

### BRIEF DESCRIPTION OF DRAWINGS

FIG. 1 is a flow chart of a method of the present disclosure.

FIG. 2 is a structural diagram of a system of the present disclosure.

FIG. 3 is a structural diagram of a plasma jet reactor.

FIG. 4 is a schematic diagram of a multi-stage pressurization system.

In the figures: 1, water pump; 2, first flow controller; 3, heat exchanger; 4, power source; 5, water electrolysis hydrogen production reactor; 6, H₂ storage tank; 7, first check valve; 8, second flow controller; 9, first centrifugal pump; 10, CO₂ storage tank; 11, second check valve; 12, third flow controller; 13, second centrifugal pump; 14, liquid collecting pipeline; 15, plasma jet reactor; 16, sight glass; 17, temperature and pressure sensors; 18, disk-shaped base; 19, gas inlet; 20, plasma jet reaction tower; 21, mixed gas intermediate storage tank; 22, third check valve; 23, fourth flow controller; 24, third centrifugal pump; 25, multi-stage pressurization system; 26, catalyst; 27, methanol synthesis reactor; 28, gas-liquid desuperheater; 29, flash tank; 30, separator; 31, methanol rectification tower; 32, methanol storage tank; 2-1, outer electrode; 2-2, inner electrode; 2-3, base; 3-1, first inlet; 3-2, gas-liquid separator; 3-3, cooling pump; 3-4, needle valve; 3-5, drying pipe; 3-6, first three-way valve; 3-7, second inlet; 3-8, second three-way valve; 3-9, first buffer tank; 3-10, diaphragm pump; 3-11, second buffer tank; 3-12, first booster pump; 3-13, first micrometering valve; 3-14, first back pressure valve; 3-15, third buffer tank; 3-16, pressure stabilizing valve; 3-17, fourth buffer tank; 3-18, second booster pump; 3-19, second micrometering valve; 3-20, second back pressure valve; and 3-21, fifth buffer tank.

### DESCRIPTION OF EMBODIMENTS

The detailed description of the present disclosure is further described in detail below in combination with the accompanying drawings.

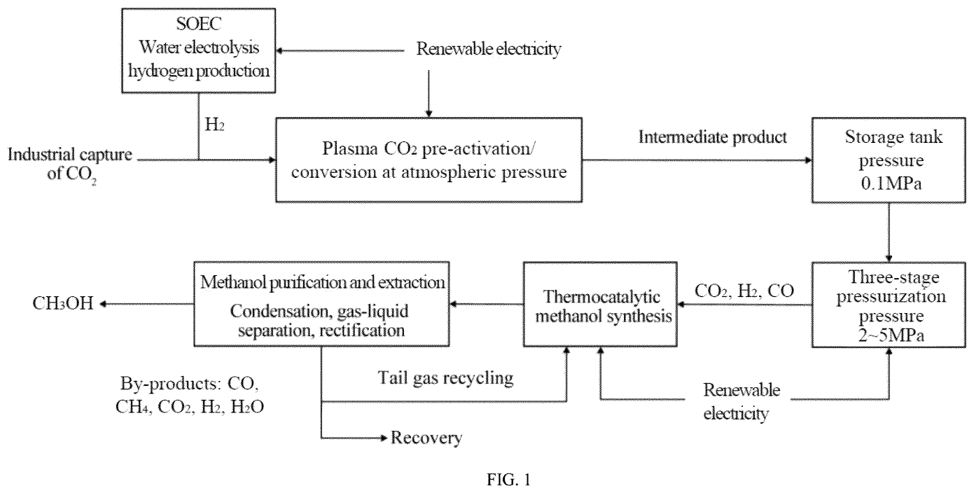

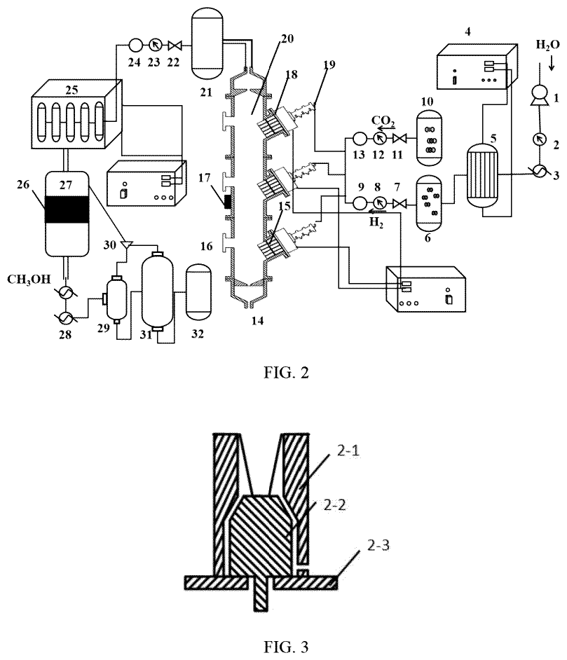

As shown in FIG. 1 and FIG. 2, an embodiment of the present disclosure provides system for utilizing renewable electricity by methanol synthesis via plasma-catalysis CO₂ hydrogenation, and the system includes reactors, a feed system, a storage and pressurization system, a power source and a purification treatment system.

The reactors include a water electrolysis hydrogen production reactor 5, a plasma jet reactor 15 and a methanol synthesis reactor 27, and all the reactors are equipped with temperature monitoring devices.

The water electrolysis hydrogen production reactor 5 is a solid oxide electrolytic cell (SOEC) reaction chamber, with a working temperature of 600° C. to 1000° C., and a main structure includes a cathode, an anode and an electrolyte layer. The cathode uses Ni/YSZ porous metal ceramic, the anode is a rare-earth-element containing perovskite (ABO₃) oxide, and the electrolyte layer is an oxygen ion conductor (YSZ or ScSZ, etc.).

In the water electrolysis hydrogen production reactor 5, H₂ is introduced through a hydrogen outlet pipe into a storage tank to be stored, the storage tank is connected with a first check valve 7, a second flow controller 8 and a first centrifugal pump 9 in sequence, and oxygen is discharged by an oxygen outlet pipe.

As shown in FIG. 3, the plasma jet reactor 15 includes a high voltage electrode and a grounding electrode, a plasma discharge is formed, and a plasma jet reaction tower 20 is connected with a mixed gas intermediate storage tank 21 which is used for storing mixed gas produced after plasma CO₂ pre-activation conversion; and the plasma jet reactor includes an outer electrode 2-1, an inner electrode 2-2, a CO₂/H₂ air inlet and a base 2-3.

The outer electrode and the inner electrode are connected to a frequency-adjustable high voltage alternating current power source with frequency of 5 to 40 kHz, a maximum output voltage of 20 kV and maximum power of 1 kW.

The outer electrode, fixed to an insulating base, is a hollow sleeve electrode with a tapered outlet at an upper portion.

The inner electrode, conical in shape, is positioned at the lower-mid part of the hollow structure of the outer electrode, and integrally formed by a lower cylinder and an upper cone frustum. The inner electrode is fixed to the base and connected to a high voltage plasma power source via an electrode lead penetrating through the base; an outer wall of the inner electrode is parallel to an inner wall of the outer electrode.

The H₂/CO₂ inlet is located at the bottom of the plasma jet reactor, and is tangentially introduced from the bottom to form a swirling rising flow in a gap between the inner and outer electrodes, so as to drive an arc between the inner and outer electrodes to rise rotationally, and plasma is ejected in a jet form under the action of a tapered outlet.

Further, the plasma jet reactors 15 are distributed and fixed on a disk-shaped base 18 in a symmetric array to form a reaction group, three reaction groups are evenly distributed on the same side of the plasma jet reaction tower 20 in a vertical staggered mode, three sight glasses 16 are correspondingly distributed on the other side and configured to observe a reaction process, a gas collecting unit is located at the top of the reaction tower, and liquid flows out from a bottom liquid collecting pipeline 14. Temperature and pressure sensors 17 are mounted on a wall surface of the reaction tower to monitor a state of the reaction system in real time.

The feed system is connected with a flow control device, and the feed system includes a vapor feed system and a CO₂ feed system.

Further, a water pump is configured to supply water to a vapor producing device, and produced vapor enters the water electrolysis hydrogen production reactor through a vapor inlet valve and a vapor inlet pipe. The vapor feed system includes the water pump 1, the vapor producing device (heat exchanger 3) and a first flow controller 2. The water pump is connected with the heat exchanger through the first flow controller and supplies water, the heat exchanger achieves heat exchange between a product methanol and a reactant water, water is heated while methanol is cooled, and the produced vapor enters the water electrolysis hydrogen production reactor 5 through the vapor inlet valve and the vapor inlet pipe.

The CO₂ feed system is configured to feed a reactant CO₂, after being mixed with H₂, the CO₂ is introduced into the plasma jet reactor 15 from a gas inlet 19 to feed CO₂. In the present embodiment, the CO₂ feed system includes a CO₂ storage tank 10, a second check valve 11, a third flow controller 12 and a second centrifugal pump 13.

The CO₂ storage tank is configured to store CO₂, and the second check valve, the third flow controller and the second centrifugal pump are connected with one another to be used for controlling the flow of CO₂.

The power source includes a water electrolysis hydrogen production power source 4, a plasma jet reactor power source and a multi-stage pressurization system power source, all powered by off-grid renewable energy (such as photovoltaic power and wind power).

The water electrolysis hydrogen production power source is configured to supply renewable electricity to drive the electrochemical hydrogen evolution reaction in the water electrolysis hydrogen production reactor 5, finally completing the production of methanol under atmospheric pressure with the plasma jet reactor as the core. The water electrolysis hydrogen production power source is a direct current device. One end of a negative electrode line is connected with a hydrogen evolution pole of the water electrolysis hydrogen production reactor, and the other end is connected with a negative electrode of the direct current power source; and one end of a positive electrode line is connected with an oxygen evolution pole of the water electrolysis hydrogen production reactor, and the other end is connected with a positive electrode of the direct current power source.

The multi-stage pressurization system power source is configured to supply power to a booster pump, an electromagnetic valve and the like in a multi-stage pressurization system 25.

The storage and pressurization system is configured to store and pressurize the CO/CO₂/H₂ mixed gas produced by the plasma jet reactor. The storage and pressurization system includes a mixed gas intermediate storage tank 21, a third check valve 22, a fourth flow controller 23, a third centrifugal pump 24 and the multi-stage pressurization system 25.

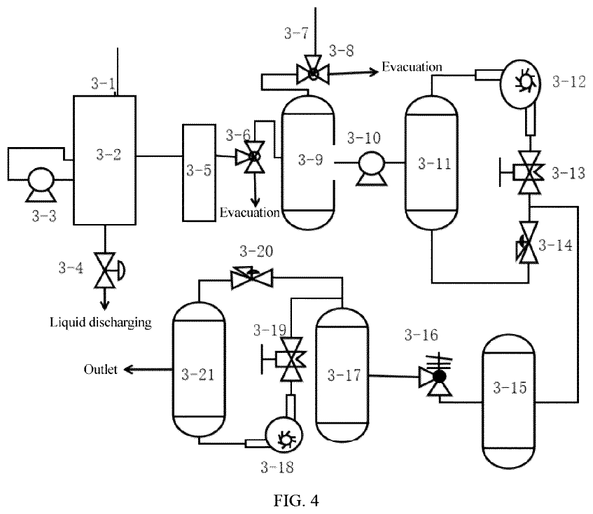

A pressurization flow chart of the multi-stage pressurization system is as shown in FIG. 4, gas enters a gas-liquid separator 3-2 after being introduced from a first inlet 3-1, separated liquid is discharged by a needle valve 3-4, and the gas is subjected to multi-stage pressurization after moisture is desorbed through a drying pipe 3-5; the gas passes a diaphragm pump 3-10 and enters a second buffer tank 3-11 from a first buffer tank 3-9 to complete front-stage pressurization; gas in the second buffer tank 3-11 is pressurized through a first booster pump 3-12 and enters a third buffer tank 3-15, if a pressure of the third buffer tank 3-15 is higher than a designated pressure, the pressure may be relieved to the second buffer tank through a first back pressure valve 3-14, the second buffer tank 3-11 and the third buffer tank 3-15 achieve parallel pressurization together with the booster pump and the back pressure valve, a fourth buffer tank 3-17 and a fifth buffer tank 3-21 further perform parallel pressurization, and the four buffer tanks complete rear-stage secondary pressurization together; and if a pressure of the first buffer tank 3-9 is too low, gas may be supplemented through a second inlet 3-7, and if a pressure of the first buffer tank 3-9 is too high, gas may be discharged through a first three-way valve 3-6.

The purification treatment system is configured to separate methanol from the remaining reaction gas and purify the methanol product. A gas-liquid mixture produced after reaction in the methanol synthesis reactor is cooled through the heat exchanger 3 and a gas-liquid desuperheater 28 in sequence, gas-liquid separation is performed on the cooled gas-liquid mixture in a flash tank 29, separated liquid methanol is purified through a methanol rectification tower 31 and then stored in a methanol storage tank 32, gas separated by the flash tank and gas separated by the rectification tower pass through a separator 30, then part of the gas is collected and then treated uniformly, and the other part of the gas is returned to the methanol synthesis reactor 27 for further reaction.

In the present disclosure, two key reactions-plasma CO₂ pre-activation/conversion and catalytic methanol synthesis-occur in two independent and isolated reaction chambers. Unlike traditional methanol synthesis methods, which rely on the direct thermocatalytic conversion of CO₂, this method, utilizing plasma-catalysis CO₂ hydrogenation, allows for these reactions to occur in separate reactors under different conditions. This separation enables the optimization of each step independently to achieve the best overall performance of the system. The inclusion of an intermediate storage system decouples the methanol synthesis reactor from the plasma jet reactor, ensuring that the methanol synthesis process remains unaffected by fluctuations in the plasma jet reactor. Meanwhile, CO₂ is partially converted to CO by adopting plasma technology. This process eliminates the need for a catalyst, thereby addressing issues of catalyst deactivation and high reaction temperatures associated with traditional thermocatalytic reverse water-gas shift reactions. Furthermore, the system allows for the adjustment of product component ratios by regulating gas flow, power, and other parameters, enabling more efficient catalytic synthesis of methanol from the CO/CO₂/H₂ mixed gas, which significantly enhances the overall process efficiency.

An embodiment of the present disclosure provides a method for utilizing renewable electricity by methanol synthesis via plasma-catalysis CO₂ hydrogenation, and the method includes the following steps:

1, The power source 4 is activated to perform water electrolysis hydrogen production, and produced hydrogen is directly introduced into the H₂ storage tank 6 for storage.

2, The CO₂ storage tank 10 and the H₂ storage tank 6 are opened, the gases are mixed in a specific ratio of 1:3 and introduced them into the plasma jet reactor 15, a swirling flow is formed inside the reactor, the power source is activated to form an arc between the inner and outer electrodes which then rises rotationally, plasma jet is ejected under the action of a tapered outlet. The reactor's power and gas flow are adjusted for optimal performance, within a range of 50 to 300 W and a range of 1 to 10 L/min, respectively, while real-time temperature monitoring is conducted. CO₂ undergoes pre-activation and the following reaction occurs:

CO₂+H₂═CO+H₂O.

3, The mixed gas produced after the reaction rises after passing an air distributor, and is introduced into a mixed gas intermediate storage tank 21 to be stored. Water flows out from a lower liquid collecting pipeline.

4, After being pressurized to 3-5 MPa through a multi-stage pressurization system, gas in the mixed gas intermediate storage tank 21 is introduced into the methanol synthesis reactor 27 to synthesize methanol over a catalyst. The specific pressurization data are as follows:

| | First-stage | Second-stage | Third-stage |
|---|---|---|---|
| Initial pressure | pressurization | pressurization | pressurization |
| 0.1 MPa | 0.5 MPa | 3 MPa | 10 MPa |

5, A gas-liquid mixture of the methanol produced after reaction in the methanol synthesis reactor 27 and unreacted gas is cooled through a heat exchanger 3 and a gas-liquid desuperheater 28 in sequence, gas-liquid separation is performed on the cooled gas-liquid mixture by using a flash tank 29, separated liquid methanol is purified through a methanol rectification tower 31 and then sent to a methanol storage tank 32, a pressure in the methanol rectification tower 31 is controlled to be 0.15 MPa, separated gas passes through a separator 30, then part of the gas is collected and then treated uniformly, and the other part of the gas is returned to the methanol synthesis reactor 27 for further reaction.

The above description is only a preferred implementation of the present disclosure, and although the present disclosure has been disclosed as above through preferred embodiments, it is not intended to limit the present disclosure. Any person skilled in the art may use the method and technical content disclosed above to make many possible changes and modifications to the technical solution of the present disclosure, or modify it into equivalent embodiments with equivalent changes, without departing from the scope of the technical solution of the present disclosure. Therefore, any simple amendments, equivalent changes, and modifications made to the above embodiments in accordance with the technical essence of the present disclosure that do not depart from the content of the technical solution of the present disclosure still fall within the scope of protection of the technical solution of the present disclosure.

What is claimed is:

1. A system for utilizing renewable electricity by methanol synthesis via plasma-catalysis CO₂ hydrogenation, comprising:
a feed system configured to provide H₂ and CO₂ for a plasma jet reactor;
a plasma jet reaction tower configured to pre-activate and convert CO₂ using plasma jet to allow CO₂ and H₂ undergo reverse water-gas shift reaction, and produce a CO/CO₂/H₂ mixed intermediate product at atmospheric pressure and low temperature;
a storage and pressurization system comprising a mixed intermediate product storage tank and a multi-stage pressurization system and configured to store and pressurize the CO/CO₂/H₂ mixed intermediate product produced by the plasma jet reactor to achieve decoupling of the plasma jet reactor and a methanol synthesis reactor;
the methanol synthesis reactor configured to catalytically synthesize methanol from the CO/CO₂/H₂ mixed intermediate product conveyed by the storage and pressurization system;
a power source configured to supply renewable electricity to the plasma jet reactor, the methanol synthesis reactor and the feed system; and
a purification treatment system configured to separate methanol from remaining reaction gas and purify the methanol.

2. The system according to claim 1, wherein the feed system comprises a water electrolysis hydrogen production reactor, a H₂ storage tank, a CO₂ storage tank, a water pump, a first flow controller and a heat exchanger, and wherein the water pump, the first flow controller and the heat exchanger are configured to convey H₂O to the water electrolysis hydrogen production reactor and control flow, and the water electrolysis hydrogen production reactor is connected to the H₂ storage tank.

3. The system according to claim 2, wherein each output of the H₂ storage tank, the CO₂ storage tank and the mixed intermediate product storage tank is provided with a check valve, a flow controller and a centrifugal pump.

4. The system according to claim 1, wherein a top of the plasma jet reaction tower is provided with a gas collecting unit that directs the mixed intermediate product to the mixed intermediate product storage tank, a bottom of the plasma jet reaction tower is provided with a liquid collecting pipeline, three plasma jet reaction groups are evenly provided at a same side of a tower body of the plasma jet reaction tower in a vertical staggered mode, and each plasma jet reaction group comprises six plasma jet reactors symmetrically arranged in a hexagonal array and fixed to a disk-shaped base; and three sight glasses are correspondingly provided at another side of the tower body of the plasma jet reaction tower and configured to observe a reaction process, and temperature and pressure sensors are provided at a wall surface of the plasma jet reaction tower.

5. The system according to claim 4, wherein the plasma jet reactor comprises:
the base;
an outer electrode fixed to the base and is a hollow sleeve electrode with a tapered outlet at an upper portion;
an inner electrode, conical in shape, arranged at a lower-mid part of a hollow structure of the outer electrode, and integrally formed by a lower cylinder and an upper cone frustum, and a bottom of the inner electrode is fixed to the base and connected to a high voltage end of a plasma power source via an electrode lead penetrating through the base; an outer wall of the inner electrode is parallel to an inner wall of the outer electrode; and
a H₂/CO₂ inlet formed in a wall surface of the outer electrode and provided at a bottom of the plasma jet reactor and tangentially introduced from the bottom of the plasma jet reactor to form a swirling rising flow in a gap between the inner electrode and the outer electrode, to drive an arc between the inner and outer electrodes to rise rotationally, and plasma is ejected in a jet form under the action of the tapered outlet; and the outer and inner electrodes are connected to a frequency-adjustable high voltage alternating current (AC) power source.

6. The system according to claim 1, wherein the methanol synthesis reactor is provided with a CO/CO₂ hydrogenation methanol synthesis reaction unit and a crude methanol outlet, CO/CO₂ and H₂ are selectively converted into crude methanol over copper-based composite nanocatalyst, the crude methanol is purified through the purification system, thereby realizing efficient chemical energy storage of power/valley electricity and collaborative conversion of greenhouse gas CO₂.

7. The system according to claim 1, wherein the multi-stage pressurization system comprises a booster pump, a buffer tank, a back pressure valve and a real-time pressure monitoring system.

8. The system according to claim 1, wherein the purification treatment system further comprises a reheater, a gas-liquid desuperheater, a flash tank, a methanol rectification tower and a methanol storage tank connected to one another in sequence, and wherein a liquid channel of the flash tank is directly connected to the methanol rectification tower, and a gas channel of the flash tank is connected to the methanol synthesis reactor and the methanol rectification tower.

9. The system according to claim 1, wherein the power source comprises a water electrolysis hydrogen production power source, a plasma jet reactor power source and a multi-stage pressurization system power source configured to supply power to a water electrolysis hydrogen production reactor, the plasma jet reactor and the multi-stage pressurization system, respectively, all powered by off-grid renewable energy.

10. A method for utilizing renewable electricity by methanol synthesis via plasma-catalysis CO₂ hydrogenation using the system according to claim 1, comprising:
step 1, activating a power source to perform water electrolysis hydrogen production, and directly introducing produced hydrogen into a H₂ storage tank for storage;
step 2, opening a CO₂ storage tank and the H₂ storage tank, mixing CO₂ and H₂ according to a specific ratio and introducing the CO₂ and H₂ into the plasma jet reactor, forming a swirling flow inside the plasma jet reactor, activating the power source to form an arc between inner and outer electrodes to rise rotationally, ejecting plasma jet under an action of a tapered outlet, adjusting power and gas flow of the plasma jet reactor for optimal performance, and conducting real-time temperature monitoring; and performing pre-activation conversion on CO₂, and reacting as follows:
CO₂+H₂═CO+H₂O,
step 3, introducing mixed CO, CO₂ and H₂ gas into the mixed gas intermediate storage tank for storage, as the mixed CO, CO₂ and H₂ gas produced by the reaction rising after passing an air distributor, and letting water flow out from a lower liquid collecting pipeline;
step 4, pressurizing, by a multi-stage pressurization system, the mixed CO, CO₂ and H₂ gas in the mixed intermediate product storage tank to 3 MPa-5 MPa, and introducing the mixed intermediate product from the mixed intermediate product storage tank into the methanol synthesis reactor to synthesize methanol under the action of a catalyst; and
step 5, cooling, by a heat exchanger and a gas-liquid desuperheater, a gas-liquid mixture of the methanol produced after reaction in the methanol synthesis reactor and unreacted gas sequentially, performing gas-liquid separation on the cooled gas-liquid mixture using a flash tank, purifying, by a methanol rectification tower, separated liquid methanol, sending the purified liquid methanol to a methanol storage tank, passing separated gas through a separator, collecting and uniformly treating a part of the separated gas, and returning another part of the separated gas to the methanol synthesis reactor for further reaction.
